(19) United States
(12) Patent Application Publication
(10) Pub. No.: US 2023/0021570 A1
(43) Pub. Date: Jan. 26, 2023

(54) RADIOTHERAPY TREATMENT DECISION METHOD AND SYSTEMS FOR PALLIATIVE CARE

(71) Applicant: KONINKLIJKE PHILIPS N.V., EINDHOVEN (NL)

(72) Inventors: Andreia Maria Araujo TRINDADE RODRIGUES, VELDHOVEN (NL); Pedro Jorge DA SILVA RODRIGUES, VELDOVEN (NL)

(21) Appl. No.: 17/786,758

(22) PCT Filed: Dec. 16, 2020
(86) PCT No.: PCT/EP2020/086355

§ 371 Date: Jun. 17, 2022

Related U.S. Application Data
(60) Provisional application No. 62/951,565, filed on Dec. 20, 2019.

(51) Int. Cl. G16H 20/40; G16H 50/30

(52) U.S. Cl. CPC G16H 20/40; G16H 50/30

(57) ABSTRACT

A non-transitory computer readable medium (26) stores instructions executable by at least one electronic processor (20) to perform a radiation therapy (RT) treatment decision method (100) that includes: calculating (102) an initial score (30) for a pre-interventional patient for each RT option of a set of RT options (32), wherein the initial score for each RT option is indicative of likelihood of discontinuation of RT in accordance with that RT option; displaying (104) the initial scores and, via a user interface (28), receiving a selection of at least one RT option from the set of RT options; for each selected RT option: optimizing (106) a RT plan (34) for the patient in accordance with the selected RT treatment option; computing (108) one or more toxicity metrics (36) for the optimized RT plan, and calculating (110) a final score (38) based on the one or more toxicity metrics for the optimized RT plan, the final score being indicative of likelihood of discontinuation of the RT treatment in accordance with the optimized RT plan; and displaying (112) the final score for the at least one selected RT option and, via the user interface, receiving a selection of a final RT option.

### FIELD

The following relates generally to the radiotherapy (RT) arts, palliative care arts, radiotherapy treatment plan selection arts, and related arts.

### BACKGROUND

Palliative radiotherapy (PRT) is the treatment of choice to control or reduce symptoms associated to advanced incurable cancer arising from primary tumor or metastasis. This treatment modality brings benefits for the patient in improving their quality of life while keeping a limited treatment burden on hospital visits and side effects. However, radiation oncologists who decide on the treatment plan should select a prescribed dose, fractionation schema and regime, modality to be used, and consider potential benefits of augmenting PRT with surgical intervention. This process of selecting an appropriate PRT regimen (or possibly no PRT at all) is a particularly challenging task with respect to advanced cancer patients since in order to tailor the treatment plan a good understanding on the current physical status and life expectancy of the patient is crucial to make the best decisions and provide the patient with the maximum benefit.

Studies have demonstrated that clinicians have limited ability to predict survival and are too optimistic in assessing the life expectancy of patients with advanced cancer. Reported accuracy of clinician's predictions regarding life expectancy is approximately 25%. As a consequence, patient selection for PRT is often suboptimal and can lead to as many as 40% to 50% of patients prematurely discontinuing their treatment before reaching completion (see, e.g., Lindsay L. Puckett, Eric Luitweiler, Louis Potters, and Sewit Teckie Journal of Oncology Practice 2017 13:9, e782-e791). Other aspects may contribute to the suboptimal decisions of PRT such as persuasion from patients and care givers to continue to treat the patient at a declared end-of-life phase, when there is a high likelihood of death in the coming days or weeks.

Strategies to improve treatment outcomes sometimes involve more aggressive regimen, but this commonly also increases severity and duration of side effects which can negatively influence quality of life in patients that are often physically weak at this stage. It is desirable to discriminate between patients that may benefit from a specific PRT intervention from the ones that will no longer benefit from it and even may have a worse outcome if administered PRT.

Currently the choice of the radiotherapy treatment for each particular patient is based on general guidelines (for example, the National Comprehensive Cancer Network (NCCN) in the United States) that only take into account tumor stage and physical condition of a patient. These guidelines are developed for groups of patients and thus lead to over-treatment in some patients and inadequate therapy in others resulting in major expense for individuals and society. This is particularly relevant for advanced cancer patients in view of their very complex and unstable status and to whom factors such as psychological, emotional, and practical/family considerations besides the patient's physical status may play a role in the capability for the patient to comply with the prescribed treatment and take full benefit.

The following discloses certain improvements to overcome these problems and others.

### SUMMARY

In one aspect, a non-transitory computer readable medium stores instructions executable by at least one electronic processor to perform a RT treatment decision method that includes: calculating an initial score for a pre-interventional patient for each RT option of a set of RT options, wherein the initial score for each RT option is indicative of likelihood of discontinuation of RT in accordance with that RT option; displaying the initial scores and, via a user interface, receiving a selection of at least one RT option from the set of RT options; for each selected RT option: optimizing a RT plan for the patient in accordance with the selected RT treatment option; computing one or more toxicity metrics for the optimized RT plan, and calculating a final score based on the one or more toxicity metrics for the optimized RT plan, the final score being indicative of likelihood of discontinuation of the RT treatment in accordance with the optimized RT plan; and displaying the final score for the at least one selected RT option and, via the user interface, receiving a selection of a final RT option.

In another aspect, an apparatus includes: a display device; at least one user input device; and at least one electronic processor programmed to: receive physiological and emotional state data of a patient that is to undergo RT; calculate an initial score indicative of a physiological and emotional state of the patient from the received data; determine at least one RT treatment regimen from a set of RT treatment regimens for the patient based on the calculated initial score; calculate a final score based on the determined RT regimen and RT treatment parameters; and select a final RT treatment regimen based on the calculated final score.

In another aspect, a RT treatment decision method includes: collecting physiological and emotional state data of a patient to be treated with RT; calculating an initial score indicative of a physiological and emotional state of the patient; determining at least one RT treatment regimen from a set of RT treatment regimens for the patient based on the calculated initial score; calculating a final score based on the determined RT regimen and RT treatment parameters; selecting a final RT treatment regimen based on the calculated final score; and displaying at least one of the final score and the selected RT treatment regimen on a display device.

One advantage resides in selecting a RT plan for a patient based on physiological and emotional state data of a patient that is to undergo RT.

Another advantage resides in accurately predicting a survival of a terminally ill cancer patient.

Another advantage resides in selecting an appropriate RT plan for a patient based on a survival likelihood of the patient during the course of the RT plan.

Another advantage resides in reducing a number of RT sessions for a patient based on a survival likelihood of the patient during the course of the RT plan.

Another advantage resides in selecting a RT plan for a patient based on a maximum benefit and a reduction in probability of the patient discontinuing the RT plan.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an illustrative apparatus for performing an RT treatment decision method or process in accordance with the present disclosure.

FIG. 2 shows exemplary flow chart operations of the apparatus of FIG. 1.

FIGS. 3-5 show examples of trend lines generated by the apparatus of FIG. 1.

### DETAILED DESCRIPTION

The following pertains to palliative radiation therapy (RT), in which the goal is to improve the patient's quality of life rather than attempting to cure the cancer. For palliative radiation therapy it is particularly important to balance the benefits provided against the costs in terms of toxicities introduced by the radiation therapy. RT toxicities can lead to weakening of the patient, more pain, and earlier decease. One important decision to be made is the choice of treatment regime, e.g.: (1) full RT in which the patient receives a full number of RT fractions; (2) hypofractional therapy in which the patient receives a reduced number of RT fractions; (3) single dose therapy; or (4) no RT. Other potential choices relate to the extent to which organ sparing may be employed.

Conventionally, the oncologist usually decides the palliative RT regimen based on the tumor stage and the patient's pre-interventional condition. In so doing, it is difficult to take into account degradation in patient condition that may be caused by toxicities introduced by the RT. Moreover, the benefits of palliative RT may not manifest until a week or more after the RT begins, whereas some toxicities manifest immediately while others are delayed, and so it is difficult to assess the benefit/cost ratio prior to commencement of RT or even during RT.

In some embodiments disclosed herein, approaches for providing pre-interventional guidance for selecting the optimal palliative RT regime are provided. The system components include an information-gathering stage (e.g. electronic questionnaire or other graphical user interface (GUI) filled out by the patient or a caseworker, possibly augmented by mining of the patient's Electronic Medical Record), and a scoring component that computes patient-specific scores for the different palliative RT regimes.

In one illustrative embodiment, the scoring component employs probability of treatment discontinuation as the scoring metric. Here the “discontinuation” may be due to the oncologist deciding to take the patient off RT, the patient deciding to stop RT, or the patient dying before the RT is complete. Advantageously, statistical treatment discontinuation data are available as a function of pre-interventional patient condition as measured by a performance status metric such as the published Karnofsky Performance Status which suitably serves as input to statistical models of the probability of treatment discontinuation for each treatment regime (full, hypofractional, single dose, or no RT).

In one illustrative approach, RT toxicities are taken into account as follows. The patient is initially scored for each RT regime without taking into account RT toxicities. These initial scores form the basis for an initial choice of candidate RT regimes (i.e., a set of RT options). The RT dose optimization is then performed for each candidate RT regime, and the predicted toxicities are quantified, for example as standard Normal Tissue Complication Probability (NTCP) values for various types of toxicities. These are then fed back into the scoring component which updates the scores for the candidate RT regimes to include the impact of the toxicities. The oncologist then makes a final selection of the RT regime based on these updated scores.

While selection of a RT regime (full, hypofractional, single fraction, or no RT) is the illustrative example, the disclosed approaches could be used optimizing other palliative RT options. For example, sparing irradiation of the thoracic spinal vertebrae can reduce hematoxicities. Hence, the patient can be scored with two or more sparing approaches taken for the spinal Organ At Risk (OAR). Furthermore, the hypofractional RT regime could be divided up into two or more groups (e.g. if full RT is 30 fractions, then the hypofractional regime could be divided into, say, 20 fractions or 10 fractions).

Optionally, the assessment/scoring may be repeated at some point during the course of the RT treatment if the patient's condition markedly changes.

An issue with performing dose optimization as part of the palliative RT selection process is the high computational cost of the dose optimization. The disclosed approach can, in some embodiments, reduce this computational cost by excluding one or more possible RT regimes prior to the dose optimization. For example, if the patient's condition is too poor then full RT may be excluded based on the initial scoring. A further approach might be to perform the dose optimizations for the score updating using a coarser optimization (e.g., with coarse voxel size, coarse angular resolution in the case of tomographic RT, and/or so forth).

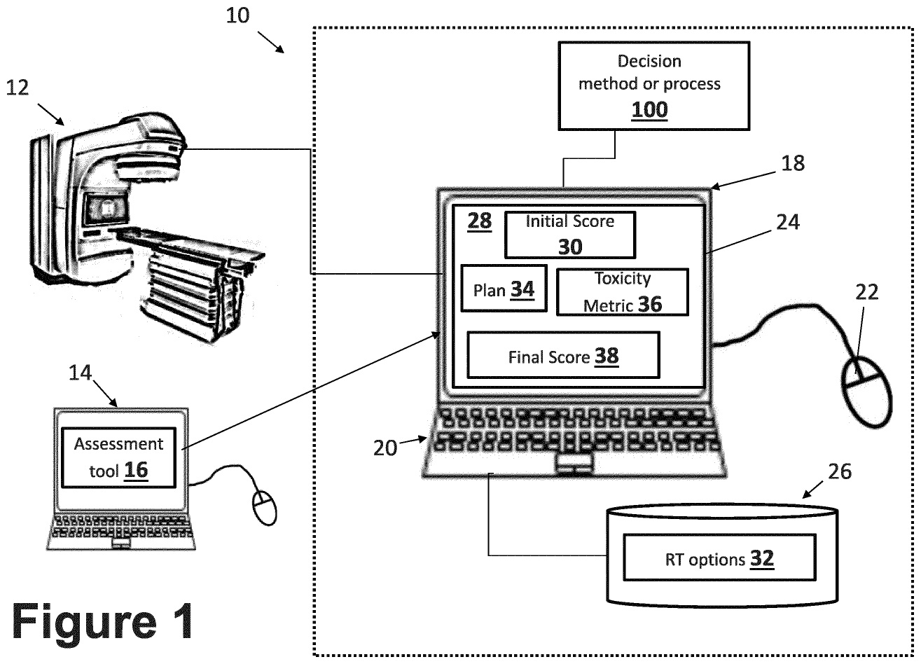

With reference to FIG. 1, an illustrative apparatus 10 for performing an RT treatment decision method or process is diagrammatically shown. The apparatus 10 can be used in conjunction with an associated RT device 12. The RT device 12 can be any type of RT device employing therapeutic radiation beams, e.g. electron beams, proton beams, high energy X-ray beams, or so forth. The RT may employ a discrete “step-and-shoot” approach in which a radiation beam source is stepped between successive fixed positions along a trajectory that partially or entirely encircles the patient. Alternatively, the RT may employ a continuous arc radiation therapy, such as VMAT, Intensity Modulated Arc Therapy (IMAT), step and short RT delivery, or so forth, in which the radiation beam source continuously irradiates the patient as the beam is revolved around the patient along a partially or entirely encircling trajectory. For example, the traversing of the trajectory comprises moving a therapeutic radiation source along a continuous arc. The number of beams may be one, two, three, or more. In the case of continuous arc radiation therapy, the number of arcs executed in the therapy session may, in general, be one, two, three, or more. For planning purposes, a continuous arc is discretized into discrete control points. In one example, the radiation delivery planning optimization system can be a linear accelerator (LINAC) with a multi-leaf collimator (MLC) configured to shape and deliver a high energy electron beam that strikes a target (e.g., an x-ray or gamma ray generator and associated hardware which serves as a radiation source) that emits x-rays (i.e., photons) in response, resulting in a therapeutic beam delivered to a patient (not shown). These are merely non-limiting illustrative examples.

The apparatus 10 for performing an RT treatment decision method or process further receives input about the patient from a patient screening device 14 which is suitably a computer, e.g., notebook computer, tablet computer, cellular telephone (cellphone), or so forth that runs an assessment tool 16 for collecting information about the patient. The assessment tool may, for example, provide a questionnaire via which information for an NCCN Distress Thermometer screening is collected. It is contemplated for the screening device 14 to be the same device as the apparatus 10; however, in some practical deployments the screening device 14 may be used by a nurse or other medical professional who carries the screening device 14 to the patient's hospital room to collect the information; or if the patient is an outpatient, the screening device 14 may be deployed in the patient's general practitioner (GP) doctor's office. As yet another potential practical deployment, the screening device 14 may be the patient's own computer or mobile device (e.g. tablet computer or cellphone) on which an application program (“app”) is loaded that provides the assessment tool 16.

As shown in FIG. 1, the apparatus 10 is suitably implemented on an electronic processing device 18, such as a workstation computer, or more generally a computer. The electronic processing device 18 typically includes a radiology reading workstation, and may also include a server computer or a plurality of server computers, e.g. interconnected to form a server cluster, cloud computing resource, or so forth, to perform more complex image processing or other complex computational tasks. The workstation 18 includes typical components, such as an electronic processor 20 (e.g., a microprocessor), at least one user input device (e.g., a mouse, a keyboard, a trackball, and/or the like) 22, and a display device 24 (e.g. an LCD display, plasma display, cathode ray tube display, and/or so forth). In some embodiments, the display device 24 can be a separate component from the workstation 18, or may include two or more display devices.

The electronic processor 20 is operatively connected with one or more non-transitory storage media 26. The non-transitory storage media 26 may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the workstation 18, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a visualization of a graphical user interface (GUI) 28 for display on the display device 24.

The apparatus 10 is configured as described above to perform a RT treatment decision method or process 100. The non-transitory storage medium 26 stores instructions which are readable and executable by the at least one electronic processor 20 to perform disclosed operations including performing the decision method or process 100. In some examples, the method 100 may be performed at least in part by cloud processing.

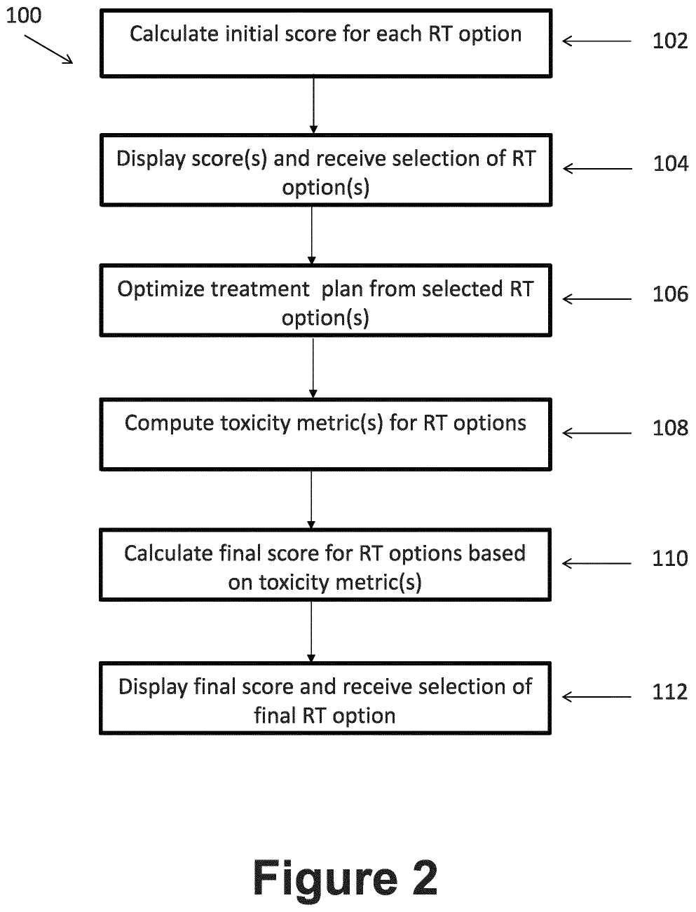

In FIG. 2, an illustrative embodiment of method 100 is diagrammatically shown as a flowchart. At an operation 102, an initial score 30 is calculated for a pre-interventional patient (that is, a patient who has not yet received the palliative RT being chosen) for each RT option of a set of RT options or regimens 32. The set of RT options can be stored in, and retrieved from, the non-transitory storage medium 26. The set of RT options 32 can include, for example, a full RT regimen option in which the patient would receive a full number of RT fractions; at least one hypofractional regimen option in which the patient would receive a number of RT fractions that is less than the full number of RT fractions; a single dose regimen option in which the patient would receive a single RT session; an option in which the patient would not receive any RT, and so forth. In the example of at least one hypofractional regimen option, there could be, for example, a full number of RT fractions being thirty fractions, then the set of RT options 32 can include a first hypofractional regimen option with fifteen fractions, a second option with five fractions, or so forth. In another example, the set of RT options can include two or more different organ at risk (OAR) sparing options. These are merely non-limiting examples, and should not be construed as limiting.

The initial score 30 for each RT option 32 is indicative of likelihood of discontinuation of RT in accordance with that RT option. In some examples, an initial score 30 with a lower numerical values indicates a higher likelihood of discontinuation, while in other examples, an initial score with a higher numerical value indicates a higher likelihood of discontinuation. In some embodiments, the calculation of the initial score 30 for each option of the set of RT options 32 is not based on any RT toxicity metric.

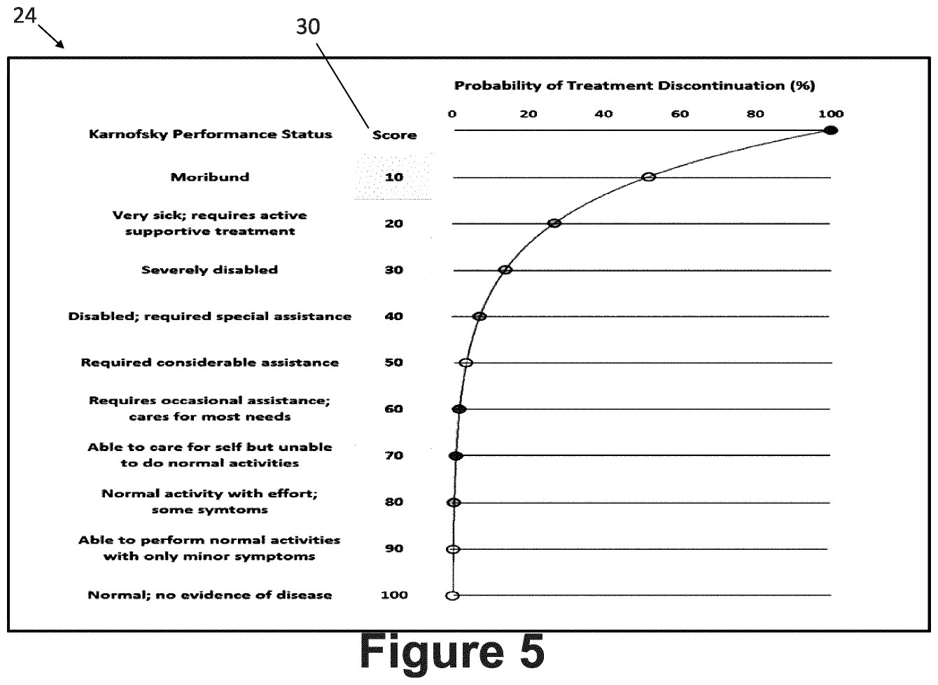

In some embodiments, the operation 102 can include collecting physiological and emotional state data of the patient from the screening assessment 16 (e.g., a health examination questionnaire, and so forth). The calculation of the initial score 30 for each RT option of the set of RT options 32 is based on the collected physiological and emotional state data. For example, the physiological and emotional state can be quantified using a Karnofsky Performance status standard in order to complete the calculation of the initial score 30. To do this, patient discontinuation statistics for cohorts of past patients who have undergone palliative RT in accord with the various RT regimens, correlated with Karnofsky Performance Status values for those patients, may be used to generate an empirical function S_{RT(opt)}(KPS) where “KPS” is the Karnofsky Performance Status and S_{RT(reg)} is the initial score 30 for the RT option denoted “opt” (e.g., “opt” may be full RT option, or single dose RT option, etc.). The initial score 30 is thus an initial estimate of likelihood of discontinuation if the RT option denoted “opt” is employed. See FIG. 5 which presents such an empirical relationship. Then, the KPS for the current patient as derived from the physiological and emotional state data of the patient collected by the screening assessment 16 is input to the function S_{RT(opt)}(KPS) for each RT option of the set of RT options 32 to obtain the patient's initial scores 32.

At an operation 104, the calculated initial scores 30 (and in some examples, the options of the set of RT options 32) are displayed on the display device 24, and an indication of selection of at least one of the RT options is received via the GUI 28. A user can select one or more of the options of the set of RT options 32 via the at least one user input device 22. For example, if the patient's physical condition is very weak as reflected by the patient information collected using the assessment tool 16 running on the screening device 14, then the medical professional may immediately discard the most aggressive option (full RT regimen), so that the full RT option is not in the set of RT options 32. Conversely, if the patient's physical condition is strong as reflected by the patient information, then the medical professional may immediately discard the least aggressive option (the single dose regimen option), so that the single dose regiment option is not in the set of RT options 32. Such early removal of RT option(s) that are plainly not appropriate for the current patient improves efficiency, because there is no need to perform dose optimization for any RT options that are removed at operation 104.

At an operation 106, a clinical RT plan 34 is optimized for the patient in accordance with the selected RT option for each selected RT treatment option. Advantageously, the operation 106 can employ existing (e.g. commercial) RT dose optimization planning tools, such as (by way of nonlimiting illustrative example) Philips Pinnacle Treatment Planning (available from Koninklijke Philips N.V.). In a typical dose optimization process, anatomical images of the current patient are acquired, for example using computed tomography (CT) or magnetic resonance (MR) imaging. The tumor to be irradiated (i.e., the target), along with organs at risk (OARs), are delineated in the anatomical images by an oncologist, dosimetrist, or other medical professional, and an attenuation or absorption map of the patient is generated from the images. Dose optimization objectives are defined by the medical professional, such as one or more objectives specifying the radiation dose to be delivered to the target (e.g. a minimum total radiation to be delivered to the entire target volume, and/or a specified fraction of the target volume to receive at least some specified minimum dose, and/or so forth) and one or more dose objectives for each OAR specifying limits or constraints on the dosage to be delivered to the OAR (e.g., maximum permissible dose analogs to the target objectives). Parameters of the RT device 12 that will deliver the radiation therapy are also defined. For example, the trajectory of the radiation source around the patient may be discretized into a set of control points (CPs), and the radiation beam profile at each CP is defined for example as a set of beamlets to be optimized. The RT dose optimization tool then iteratively optimizes the beamlets by, for each iteration, estimating the dose distribution to be delivered to the patient taking into account attenuation as indicated by the attenuation map and adjusting the RT device parameters between iterations until the estimated dose distribution matches the dose objectives to the extent practicable.

It will be appreciated that the dose optimization 106 can be highly computationally expensive. The number of parameters to be optimized can be in the hundreds or more, and each iteration entails computing the estimated dose distribution in the patient which is a sum of contributions from all beamlets of all CPs. For developing a deliverable RT plan, the dose optimization is performed at high spatial resolution (the dose distribution is represented by a map with small voxel size) and with the radiation source trajectory discretized into a fairly large number of CPs; and this high resolution increases computational time and processor load. However, for the purpose of the decision method or process 100 of the present application, the goal is more modest, namely obtaining an improved estimate of potential toxicities that may be produced by the various RT options 32. Accordingly, in some examples, the operation 106 can include coarsely optimizing the RT plan 34 for each selected RT option using a coarse spatial resolution (optionally including a coarser discretization of the radiation source trajectory into CPs).

At an operation 108, one or more toxicity metrics 36 are computed for the optimized RT plan 34. In some examples, the RT toxicity metrics 36 comprise Normal Tissue Complication Probability (NTCP) values, which are commonly computed for generated RT plans by existing commercial dose optimization tools such as Pinnacle. The NTCP value for a given toxicity risk is suitably computed using the optimized dose distribution in the OAR or OARs to which the toxicity risk relates.

At an operation 110, a final score 38 is calculated based on the one or more toxicity metrics 36 for the optimized clinical RT plan 34. The final score 38 is indicative of likelihood of discontinuation of the RT treatment in accordance with the optimized RT plan. Similar to the initial score 30, in some examples, a final score 38 with a lower numerical values indicates a higher likelihood of discontinuation, while in other examples, a final score with a higher numerical value indicates a higher likelihood of discontinuation.

In some embodiments, the calculation of the final score 38 can include determining a difference between the initial score 30 and an optimal score for at least one of the RT options 32. The determined difference is then multiplied multiplying by the computed one or more toxicity metrics 36 for the at least one optimized RT plan 34 to determine the final score 38. For example, the initial score 30 can be determined to be 50 (determined at the operation 102) and the toxicity metric 36 can be determined to be 0.6 (determined at the operation 108). If an optimal score for the patient is 20, then the final score 38 can be determined by (50−20)*(0.6) for a final score of 32.

At an operation 112, the final score 38 is displayed on the display device 24 for the at least one selected RT option 32. The user can then make a selection of a final RT option 32 is received via the GUI 28 using the at least one user input device 22. In some examples, the one or more toxicity metrics 36 can also be displayed for the at least one selected RT option 32.

In some embodiments, after receiving the selection of the final RT option 32, the clinical plan 34 can be further optimized using a clinical spatial resolution that is finer than the coarse spatial resolution. Advantageously, the coarse optimization at the operation 106 and the fine optimization after selection of the final RT option 32 allows a more efficient generation of the clinical RT plan 34 by avoiding the need to generate a clinical RT plan for each RT option. Moreover, in some embodiments the coarsely optimized dose distribution from operation 106, with suitable resampling to the higher resolution, serves as the initial dose distribution for optimizing the clinical RT plan 34 for delivery to the patient.

The clinical RT plan 34 is used for delivery of the RT treatment to the patient using the RT device 12. As is known in the art, developing the deliverable RT plan may involve converting optimization parameters to physically realizable parameters. For example, if the dose optimization employs beamlet parameters these may be converted to physically realizable multileaf collimator (MLC) settings of the RT device 12. In some embodiments, subsequent to applying at least one RT fraction to the patient, the one or more toxicity metrics 36 can be updated based on clinical assessment of the patient. For two or more RT options of the set of RT options 32 (including the final RT option), the final scores 38 can be re-calculated based on the updated one or more toxicity metrics. The updated final scores 36 can be displayed for the two or more RT options.

### Example

In this example, the method 100 is described by which a fast assessment (i.e., patient screening) and comprehensive assessments can be used and based on which different recommendations are generated to refine the RT treatment to maximize treatment benefit and quality of life and minimize the treatment discontinuation probability.

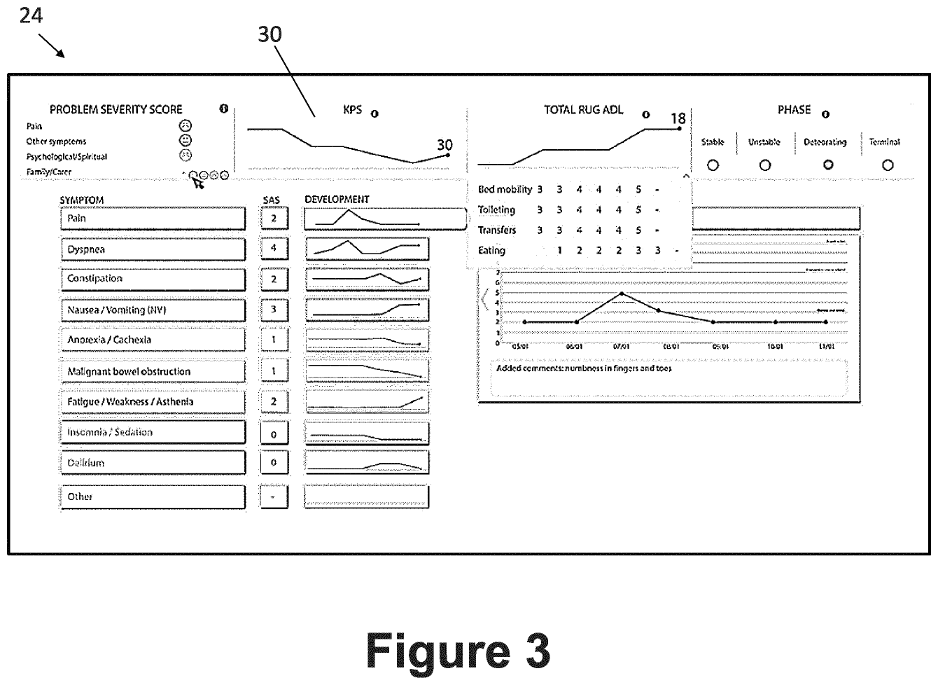

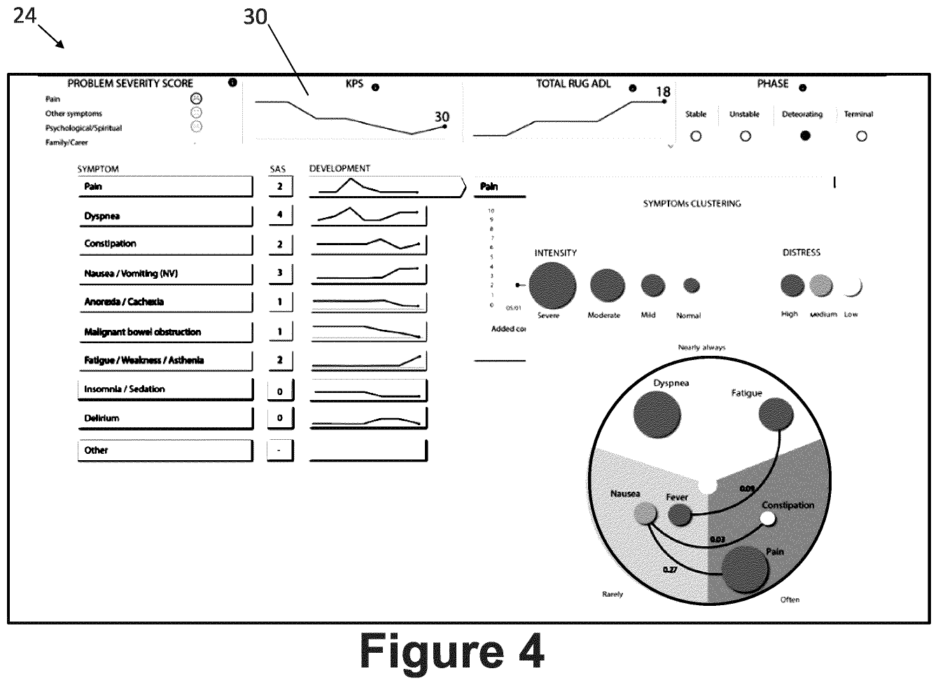

A medical professional, such as a nurse can screen a patient using a questionnaire and inputs the results in the GUI 28 at a predetermined time. The questionnaire can be, for example, a NCCN Distress Thermometer screening form. These inputs are collected, and a graphical trend is displayed on the display device, as shown in FIGS. 3 and 4.

The initial Karnofsky Performance Status score 38 is calculated using the information from the trends show in FIGS. 3 and 4, along with patient observation. The initial score 30 is used to determine the probability of RT treatment discontinuation. A graphical trend of a performance status is used in this determination. If the initial score 30 is high (i.e., a high probability of treatment discontinuation), a recommendation is made from the set of RT treatment options 32 for hypo-fractionation radiotherapy (up to a maximum of 5 fractions) or single dose radiotherapy is made. The initial score 30 can be weighted by other factors, such as example the presence of metastasis in areas sensitive to large depositions of dose, for example, a spinal cord, as shown in Table 1.

TABLE 1

| | Patient A | Patient B | Patient D |
|---|---|---|---|
| Probability of RT | 0.10 | 0.10 | 0.70 |
| treatment | | | |
| discontinuation | | | |
| High-risk | 0 | + | 0 |
| metastasis | | | |
| Score | ++ | + | −−− |
| Standard RT | | | |
| Score Hypo- | +++ | − | ++ |
| fractionation RT | | | |
| (>1 and <=5) | | | |
| Score for Single | +++ | −− | +++ |
| RT | | | |

These scores 30 and the recommended treatment option 32 are displayed on the display device 24, and the method 100 continues.

Dosimetric parameters for the chosen treatment are then extracted. Parameters for organs at risk are then extracted and can be used to update the score, as shown in Error! Reference source not found.

TABLE 2

| | Patient A | Patient B | Patient D |
|---|---|---|---|
| Probability of RT | 0.10 | 0.10 | 0.70 |
| treatment | | | |
| discontinuation | | | |
| High-risk | 0 | + | 0 |
| metastasis | | | |
| Score | ++ | ++ | −−− |
| Standard RT | | | |
| Score Hypo- | +++ | −− | ++ |
| fractionation RT | | | |
| HT score for | − | − | − |
| standard RT | | | |
| HT score for | + | + | + |
| Hypo-fractionation | | | |
| RT | | | |
| Updated Score | + | + | −−−− |
| Standard RT | | | |
| Updated Score | ++++ | − | +++ |
| Hypo-fractionation | | | |
| RT | | | |

For illustration purposes pertinent to this example, low dose radiation bath volumes (e.g., V5, V10 and mean dose to vertebra) are computed from the plan 34 and used to estimate the probability of hematoxicities. Recent results that have shown that sparing irradiation to thoracic vertebra (and consequently bone marrow) should be considered, with limits to the mean vertebra dose of around 24 Gy, to avoid hematoxicities as well as that standard radiotherapy with large fractionation (10 to 30) are directed associated with higher probability of hematoxicities and reduction of survivability in comparison with hypo-fractionation RT.

In some example embodiments disclosed herein, screening assessments can be extracted/complemented with biometric information derived from non-obtrusive measurements collected from sensors or analysis of daily voice recordings from the patient story, via a mobile interface or an artificial intelligence (AI)-assistance.

In other example embodiments disclosed herein, a correlation of RT treatment outcomes with the computed final scores 38 can be done based on cohorts of patients and threshold scores for the different radiotherapy modalities (e.g. standard versus hypo-fractionation) extracted from those patients.

In other example embodiments disclosed herein, screening and comprehensive screening assessments can be collected remotely, either by requiring the patient/informal caregiver to input the information via a mobile interface (for example: a smart phone).

In other example embodiments disclosed herein, a desired final score 38 can be added as part of the objectives for the radiotherapy optimization operation 106. The radiotherapy modality and dose distributions to organs at risk that provide the most benefit in terms of the final score 38 are selected.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory computer readable medium storing instructions executable by at least one electronic processor to perform a radiation therapy (RT) treatment decision method, the method comprising:
calculating an initial score for a pre-interventional patient for each RT option of a set of RT options, wherein the initial score for each RT option is indicative of likelihood of discontinuation of RT in accordance with that RT option;
displaying the initial scores and, via a user interface, receiving a selection of at least one RT option from the set of RT options;
for each selected RT option:
optimizing a RT plan for the patient in accordance with the selected RT treatment option;
computing one or more toxicity metrics for the optimized RT plan, and
calculating a final score based on the one or more toxicity metrics for the optimized RT plan, the final score being indicative of likelihood of discontinuation of the RT treatment in accordance with the optimized RT plan; and
displaying the final score for the at least one selected RT option and, via the user interface, receiving a selection of a final RT option.

2. The non-transitory computer readable medium of claim 1, wherein the optimizing of the RT plan for each selected RT option comprises optimizing of the RT plan for each selected RT option using a first spatial resolution, and the method further comprises:
after receiving the selection of the final RT option, optimizing a clinical RT plan for the final RT option using a second spatial resolution that is finer than the first spatial resolution.

3. The non-transitory computer readable medium of claim 1, wherein:
the calculating of the initial score for each RT option of the set of RT options (32) is not based on any RT toxicity metric.

4. The non-transitory computer readable medium of claim 3, wherein the RT toxicity metrics comprise Normal Tissue Complication Probability (NTCP) values.

5. The non-transitory computer readable medium of claim 1, wherein the calculating of the final score includes:
determining a difference between the initial score and a score for at least one of the RT options assuming a toxicity occurs due to the RT treatment option;
multiplying the determined difference by the computed one or more toxicity metrics for the at least one optimized RT plan corresponding to the assumed toxicity to determine the final score.

6. The non-transitory computer readable medium of claim 1, wherein the method further includes:
subsequent to applying at least one RT fraction to the patient in accordance with the final RT option, updating the one or more toxicity metrics based on clinical assessment of the patient and, for two or more RT options of the set of RT options including the final RT option, re-calculating the final scores based on the updated one or more toxicity metrics and displaying the updated final scores for the two or more RT options.

7. The non-transitory computer readable medium of claim 1, wherein the method further includes:
collecting physiological and emotional state data of the patient from a screening assessment, the calculating of the initial score for each RT option of the set of RT options being based on the collected physiological and emotional state data.

8. The non-transitory computer readable medium of claim 7, wherein calculating the initial score includes:
quantifying the physiological and emotional state data of the patient using a Karnofsky Performance status standard.

9. The non-transitory computer readable medium of claim 1, wherein the set of RT options includes:
a full RT regimen option in which the patient would receive a full number of RT fractions;
at least one hypofractional regimen option in which the patient would receive a number of RT fractions that is less than the full number of RT fractions;
a single dose regimen option in which the patient would receive a single RT session; and
an option in which the patient would not receive any RT.

10. The non-transitory computer readable medium of claim 1, wherein the set of RT options includes two or more different organ at risk (OAR) sparing options.

11. The non-transitory computer readable medium of claim 1, wherein the displaying of the final scores for the at least one selected RT option includes also displaying the one or more toxicity metrics for the at least one selected RT option.

12. An apparatus, comprising:
a display device;
at least one user input device; and
at least one electronic processor programmed to:
receive physiological and emotional state data of a patient that is to undergo radiation therapy (RT);
calculate an initial score indicative of a physiological and emotional state of the patient from the received data;
determine at least one RT treatment regimen from a set of RT treatment regimens for the patient based on the calculated initial score;
calculate a final score based on the determined RT regimen and RT treatment parameters; and
select a final RT treatment regimen based on the calculated final score.

13. The apparatus of claim 12, wherein the at least one electronic processor is further programmed to:
receive, via the at least one user input device, inputs of the physiological and emotional state data of the patient into a graphical user interface displayed on the display device.

14. The apparatus of claim 12, wherein the at least one electronic processor is further programmed to:
display at least one of the final score and the selected RT treatment regimen) on the display device.

15. The apparatus of claim 12, wherein the calculating of the final score includes:
determining a difference between the initial score and an optimal score for at least one of the RT options;
multiplying the determined difference by the computed one or more toxicity metrics for the at least one optimized RT plan to determine the final score.

16. The apparatus of claim 12 wherein the at least one electronic processor is further programmed to:
subsequent to applying at least one RT fraction to the patient, update the one or more toxicity metrics based on clinical assessment of the patient and, for two or more RT options of the set of RT options including the final RT regimen, re-calculating the final scores based on the updated one or more toxicity metrics and displaying the updated final scores for the two or more RT options.

17. A radiation therapy (RT) treatment decision method, comprising:
collecting physiological and emotional state data of a patient to be treated with RT;
calculating an initial score indicative of a physiological and emotional state of the patient;
determining at least one RT treatment regimen from a set of RT treatment regimens for the patient based on the calculated initial score;
calculating a final score based on the determined RT regimen and RT treatment parameters;
selecting a final RT treatment regimen based on the calculated final score; and
displaying at least one of the final score and the selected RT treatment regimen on a display device.

18. The method of claim 17, further including:
calculating the initial score for each RT treatment regimen of the set of RT treatment regimens without accounting for RT toxicities;
selecting a plurality of candidate RT treatment regimens from the set of RT treatment regimens based on the initial scores;
quantifying RT toxicities for each candidate RT treatment regimen.

19. The method of claim 18, further including:
calculating the final scores using the quantified RT toxicities; and
determining the RT treatment regimen based on the calculated final scores.

20. The method of claim 1, further including:
determining a difference between the initial score and an optimal score for at least one of the RT regiments;
multiplying the determined difference by the computed one or more toxicity metrics for the at least one optimized RT plan to determine the final score.
